(19) United States
(12) Patent Application Publication
(10) Pub. No.: US 2020/0208786 A1
(43) Pub. Date: Jul. 2, 2020

(54) METHOD FOR TRACKING AND LOCATING CONTAMINATION SOURCES IN WATER DISTRIBUTION SYSTEMS BASED ON CONSUMER COMPLAINTS

(71) Applicant: TONGJI UNIVERSITY, Shanghai (CN)

(72) Inventors: Kunlun XIN, Shanghai (CN); Lian SUN, Shanghai (CN); Hexiang YAN, Shanghai (CN); Tao TAO, Shanghai (CN); Shuping LI, Shanghai (CN); Jiaying WANG, Shanghai (CN)

(21) Appl. No.: 16/600,547

(22) Filed: Oct. 13, 2019

(30) Foreign Application Priority Data
Jan. 2, 2019 (CN) 201910002247.X

(51) Int. Cl. F17D 5/00; G01N 33/18; G06N 3/08; G06N 3/04

(52) U.S. Cl. CPC F17D 5/00; G06N 3/04; G06N 3/08; G01N 33/18

(57) ABSTRACT

The present invention relates to a method for tracking and locating a contamination source in a water distribution system based on consumer complaints, comprising following steps: S1: generating a contamination matrix by location information complained by consumers; S2: determining similarity between candidate nodes and classifying the candidate nodes; S3: adding a random complaint hysteresis time and constructing a consumer complaint sample; and S4: training, validating and testing a convolutional neural network by the consumer complaint sample, and using the convolutional neural network in practically tracking and locating a contamination source. Compared with the prior art, the present invention has the following advantages. The contamination source is located in the consumer complaint pattern, according to the real-time consumer complaints after a contamination accident occurs. The method works well in contamination source identification for both water source contamination and non-water source contamination.

### CROSS-REFERENCE TO RELATED APPLICATIONS

This application claims the benefit of priority from Chinese Patent Application No. CN 201910002247.X, filed on Jan. 2, 2019. The content of the aforementioned application, including any intervening amendments thereto, is incorporated herein by reference in its entirety.

### TECHNICAL FIELD

The present invention relates to a method for tracking and locating a contamination source, and in particular to a method for tracking and locating a contamination source in a water distribution system based on consumer complaints.

### BACKGROUND OF THE PRESENT INVENTION

The urban water distribution system is the main way for residents to obtain drinking water. The safety of the water distribution system directly affects the safety of residents in water consumption, and is also an important factor to ensure social stability and economic development. The safe water supply of the water distribution system means that, under the premise of qualified quality and sufficient volume and pressure, the finished water must meet the requirements of consumers on quality, volume and pressure in lowest possible cost. Generally, the finished water can meet the national water quality standards after water quality treatment. There are two main reasons for water quality problems in the water distribution system: (1) intrinsic water quality deterioration; and (2) sudden contaminant intrusion. Therefore, to ensure the water quality of the water distribution system, it must ensure that water will not cause short-term or long-term health hazards to the human body during use, and that the system must have good prevention, protection, emergency treatment, and recovery functions in sudden accidents such as sudden water contamination accidents, water plant operation accidents, man-made intentional damage and natural disasters.

In recent years, the water distribution systems in many cities in China are threatened by sudden water contamination accidents. Contaminants in such sudden water contamination accidents often enter the water distribution systems in an instant or short time and diffusion rapidly. This does great harm to the society. When a contamination accident occurs, it is necessary to quickly locate the contamination source in order to timely block the diffusion of contamination and repair the contaminated water distribution system. The technique of tracking and locating a contamination source is an inversion technique of deriving the position of contaminant injection nodes and other information based on the water quality information of the water distribution system together with the properties of the water distribution system. By the technique of tracking and locating a contamination source in a sudden contamination accident in a water distribution system, the location of the contamination source and the injection time can be quickly determined. Thus, rapid and active emergency treatment measures can be taken appropriately to minimize the impact and loss caused by contamination.

For cities with water quality monitoring systems, scholars both in China and abroad have proposed many effective methods to locate the contamination source. Shang, et al. proposed a particle backtracking algorithm to locate an unknown contamination source in a water distribution system in 2002. Laird, et al. proposed a nonlinear programming method to minimize the error between simulated and measured values in 2005, and improved the method in 2006 to locate multiple contamination sources. Preis and Ostfeld (2006) realized inverse modelling by a coupled model trees-linear programming algorithm, on the basis of a large number of water quality simulations. Huang and McBean (2009) used a data mining approach with maximum likelihood to identify where and when contamination occurs. Cristo, et al. (2008) achieved localization of the contamination source by minimizing the error between simulated and measured values by a proportional matrix. In 2008, Kim, et al. proposed a method for identifying pathogenic microbial contamination sources by artificial neural networks in order to isolate the contaminated areas to reduce hazards. In 2010, Propato, et al. proposed a method for determining the contamination source, by narrowing the range of the contamination source by linear algebra and obtaining the minimum relative entropy. Liu, et al. (2011) explored a dynamic optimization method based on an evolutionary algorithm to respond to contamination events in real time. Shen and McBean (2012) determined possible contamination sources by mining offline-built databases by a data mining approach and simulating multiple scenarios simultaneously. Among scholars in China, Wang Kangle (2010) tracked the contamination source in the water distribution system by a relational tree-linear programming algorithm, and obtained the location of the contamination source node and the contaminant injection properties by solving the linear programming problem. Li Hongwei, et al. (2011) located the contamination source and analyzed main factors that influence the model, by an improved simulation-optimization backtracking method, based on experimental data.

The conventional techniques of identifying a contamination source in a water distribution system are all based on the premise that the online monitored data of the water quality in the water distribution system is sufficient and accurate. At present, the accuracy of most urban water distribution system models in China cannot meet the requirements of water quality simulation. Furthermore, most of the water quality monitoring equipment is not perfect, and the monitored data is less accurate. Therefore, it is difficult for the conventional contamination source identification techniques based on monitored water quality data to be implemented. When there is no enough monitored information for research and utilization in a sudden contamination accident, the consumer complaints can be used as important information that reflects the water quality status of the water distribution system. Some scholars in China have carried out studies on methods for tracking and locating a contamination source based on consumer complaints, in which each consumer is regarded as a “water quality monitoring device”. In 2012, Xin Kunlun, et al. proposed construction of a pattern recognition neural network, based on the sequence of consumer complaints about water quality after the candidate nodes are contaminated, to determine the contaminant injection location. In 2013, Xin Kunlun, et al. proposed the comparison of the probability of complaints when different candidate contamination source nodes are contaminated, by probability theory analysis, to determine the highest possible contamination source node.

In conclusion, at present, there is no technique of locating a contamination source widely used by the China's water department, although there have been many studies on the contamination source location.

### SUMMARY OF THE PRESENT INVENTION

An objective of the present invention is to provide a method for tracking and locating a contamination source in a water distribution system based on consumer complaints, in order to overcome shortcomings in the prior art.

The purpose of the present invention may be realized by the following technical solutions.

A method for tracking and locating a contamination source in a water distribution system based on consumer complaints is provided, comprising following steps:

S1: generating a contamination matrix by location information complained by consumers;

S2: determining similarity between candidate nodes and classifying the candidate nodes;

S3: adding a random complaint hysteresis time and constructing a consumer complaint sample; and

S4: training, validating and testing a convolutional neural network by the consumer complaint sample, and using the convolutional neural network in practically tracking and locating a contamination source.

Further, the contamination matrix in the step S1 is expressed by the following formula:

$$C = [ \begin{matrix}{k}_{1 , 1} & … & {k}_{1 , n - 1} & {k}_{1 , n} \\ … & … & … & … \\ {k}_{m - 1 , 1} & … & {k}_{m - 1 , n - 1} & {k}_{m - 1 , n} \\ {k}_{m , 1} & … & {k}_{m , n - 1} & {k}_{m , n}\end{matrix} ]$$

where, C is the contamination matrix, m is the number of nodes in a water distribution system, n is the number of points complained by consumers, and k_{i,j}=0 or 1, wherein k_{i,j}=0 when contaminants are injected to the i^{th} node but the complained j^{th} node is not perceived as being contaminated, and k_{i,j}=1 when contaminants are injected to the i^{th} node and the complained j^{th} node is perceived as being contaminated, 1≤i≤m, 1≤j≤n.

Further, the determination and classification in the step S2 are done by the Chebyshev distance, expressed by the following formula:

D_{Chebyhev}(t′,T′)≤1

where, t′ and T′ each represent a relative time vector for contaminants added in two candidate contamination source nodes to reach complained nodes.

Further, the consumer complaint sample in the step S3 is a 48×n matrix containing elements 0 and 1.

Further, normalization of the 48×n matrix containing elements 0 and 1 is to normalize the position of non-zero elements so that an average value of time subscripts of all non-zero elements is 24, expressed by the following formula:

T_{i1,changed}=Tᵢ₁−T̅+24; (i1=1,2, . . . n)

where, Tᵢ₁ represents the original time subscript value of a non-zero element in the matrix, T_{i1,changed} represents the changed time subscript value of a non-zero element, and T̅ represents an average value of time subscripts of all non-zero elements.

Further, the convolutional neural network in the step S4 has hyper-parameters set as follows:

| Name | Structural parameter | Activation function |
|---|---|---|
| Input layer | 48 × n matrix | |
| Convolutional layer 1 | 3 × 3 × 8 S = 1 | ReLU |
| Convolutional layer 2 | 3 × 3 × 8 S = 1 | ReLU |
| Pooling layer | 2 × 2 S = 2 (Max pooling) | |
| Fully connected layer | 32 (neuron) | ReLU |
| Output layer | a | Softmax |

where, a represents the number of types of candidate nodes, and S represents the movement step size.

Further, the convolutional neural network in the step S4 has an initial learning rate of 0.1 and an attenuation coefficient of 0.99, uses L2 regularization in two fully connected layers at a regularization coefficient of 0.0001, and has a number of training iterations of 15000.

Compared with the prior art, the present invention has the following advantages:

(1) The method is high in accuracy, and is scientific and rational. In the present invention, the contamination source is located in the consumer complaint pattern, according to the real-time consumer complaints after a contamination accident occurs. The method works well in contamination source identification for both water source contamination and non-water source contamination, and is somewhat robust to the uncertainty of consumer complaint hysteresis time. In cities where the on-line equipment for monitoring water quality in water distribution systems is not perfect, the contamination source can be quickly located, and the city's emergency response to water quality accidents in water distribution systems can be enhanced. The method is highly practical.

(2) The calculation is fast. In the present invention, the contamination matrix is calculated by a convolutional neural network that has an initial learning rate of 0.1 and an attenuation coefficient of 0.99, uses L2 regularization in two fully connected layers at a regularization coefficient of 0.0001, and has a number of training iterations of 15000. The calculation is fast and accurate.

### BRIEF DESCRIPTION OF THE DRAWINGS

FIG. 1 is a flowchart of a method for tracking and locating a contamination source in a water distribution system based on a convolutional neural network and consumer complaints;

FIG. 2 is a sample graph of stimulating the consumer complaint pattern, according to the present invention;

FIG. 3 is a model diagram of the conventional neural network according to the present invention;

FIG. 4 is a model diagram of the topology of an exemplary water distribution systemNet3, according to the present invention; and

FIG. 5 is a curve graph showing the reduction in model training loss.

### DETAILED DESCRIPTION OF THE PRESENT INVENTION

The technical solutions in the embodiments of the present invention will be described clearly and completely with reference to the accompany drawings in the embodiments of the present invention. Apparently, the embodiments to be described are merely some embodiments of the present invention rather than all embodiments of the present invention. Based on the embodiments of the present invention, all other embodiments, obtained by a person of ordinary skill in the art without paying any creative effort, are included in the protection scope of the present invention.

### Embodiment

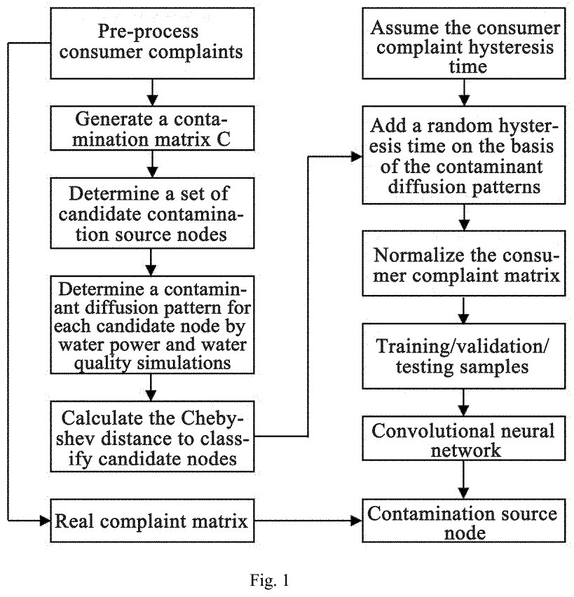

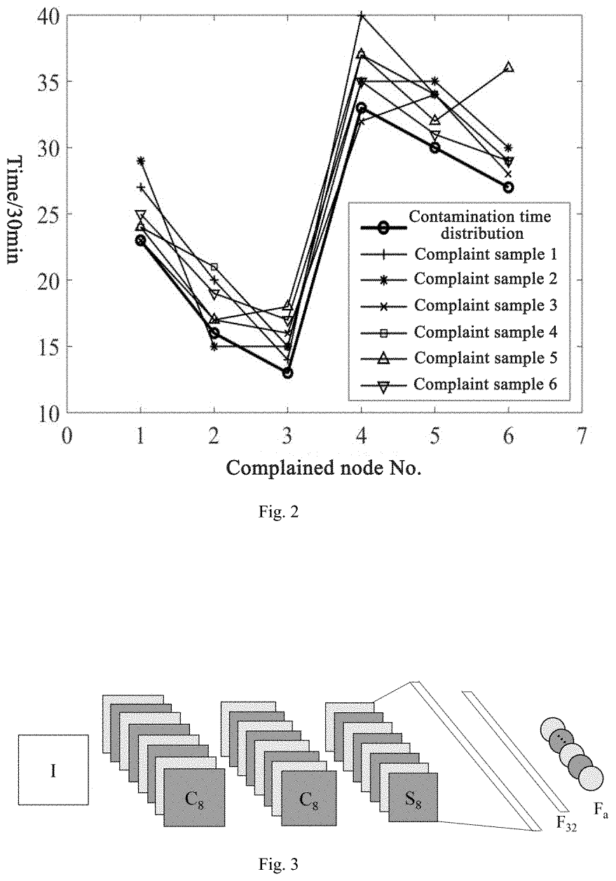

As shown in FIGS. 1, 2 and 3, a method for tracking and locating a contamination source in a water distribution system based on a convolutional neural network and consumer complaints is provided, comprising following steps:

1) generating a contamination matrix by location information complained by consumers, and obtaining a set of candidate contamination source nodes;

2) by calculating the Chebyshev distance, determining similarity between contaminant diffusion patterns for the candidate nodes and classifying the candidate nodes;

3) adding a random complaint hysteresis time on the basis of the contaminant diffusion patterns and constructing a consumer complaint sample; and

4) training, validating and testing a convolutional neural network by the consumer complaint samples that are classified into a training set, a validation set and a testing set, to assess the validity of the model, and using the convolutional neural network in tracking and locating a contamination source in a real contamination event.

The step (1) specifically comprises:

By improving the contamination matrix, only water quality information of nodes complained by consumers is extracted to obtain a contamination matrix C. A set of candidate contamination source nodes is obtained. This process is simply expressed by the following formula:

$$C = [ \begin{matrix}{k}_{1 , 1} & … & {k}_{1 , n - 1} & {k}_{1 , n} \\ … & … & … & … \\ {k}_{m - 1 , 1} & … & {k}_{m - 1 , n - 1} & {k}_{m - 1 , n} \\ {k}_{m , 1} & … & {k}_{m , n - 1} & {k}_{m , n}\end{matrix} ]$$

where, C is the contamination matrix, m is the number of nodes in a water distribution system, n is the number of points complained by consumers, and k_{i,j}=0 or 1, wherein k_{i,j}=0 when contaminants are injected to the i^{th} node but the complained j^{th} node is not perceived as being contaminated, and k_{i,j}=1 when contaminants are injected to the i^{th} node and the complained j^{th} node is perceived as being contaminated, 1≤i≤m, 1≤j≤n.

The step (2) specifically comprises:

The candidate contamination source nodes are classified by calculating the Chebyshev distance. Nodes with a similar contaminant diffusion pattern are classified into one type. Given that the relative time vectors for contaminants added in two candidate contamination source nodes to reach complained nodes are t=(t₁, t₂, . . . , tₙ) and T=(T₁, T₂, . . . , Tₙ), wherein n is the number of complained nodes, then the average values of the two vector elements are t(−)t(−)t(−)t̅ and T̅:

$$\overline{t} = \frac{1}{n} {\sum }_{i 2 = 1}^{n} {t}_{i 2} , \overline{T} = \frac{1}{n} {\sum }_{i 2 = 1}^{n} {T}_{i 2} , i 2 = 1 , 2 , … , n$$

by the following formula:

t′₃=t₂−t̅,T′ᵢ₃=Tᵢ₂−T̅, i3=1,2, . . . ,n

then: t′=(t₁′, t₂′, . . . , tₙ′) and T′=(T₁′, T₂′, . . . , Tₙ′).

If both t′ and T′ meet the following condition, the two candidate contamination source nodes are classified into a same type, or otherwise they are classified into different types:

D_{Chebyshev}(t′,T′)≤1

In the step 3), the training samples used for training and assessing the convolutional neural network are constructed in the following specific way:

At a certain point in time, contaminants of a certain concentration are injected continuously to each candidate contamination source node. The water quality at each node in the water distribution system at different moments can be obtained by stimulating the water power and water quality of the water distribution system in a delay of 24 hours. It is supposed that consumers at a node can perceive the abnormality in water quality and might make complaints only when the concentration of contaminants is higher than the reporting limit RL of the human body to contaminants. In the simulation, moments when, after contamination occurs, consumers at nodes can perceive the contamination are output as a 48×n matrix containing elements 0 and 1, for example matrix A. 48 and n respectively represent the time duration of 0-24 hours (step size: 30 min) and the number of complained nodes, wherein 1 represents there are consumer complaints at a certain moment at a certain node, and 0 represents there is no consumer complaint. On the basis of this matrix, considering that there is certain hysteresis from the earliest consumer complaint moment to the actual contamination occurrence moment, on the consumption that the hysteresis time follows the normal distribution (Δt˜N(μ,σ²)), the hysteresis is added, as noise, to the moment when contamination starts at the node, to obtain a consumer random complaint sample for each matrix.

$$1 \qquad 2 \qquad 3 \qquad … \qquad 12 \qquad 13 \qquad 14 \qquad 15 \qquad 16 \qquad … \qquad 22 \qquad 23 \qquad 24 \qquad 25 \qquad 26 \qquad 27 \qquad 28 \qquad 29 \qquad 30 \qquad 31 \qquad 32 \qquad 33 \qquad … \qquad 47 \qquad 48$$

$$A = {[ \begin{matrix}0 & 0 & 0 & … & 0 & 0 & 0 & 0 & 0 & … & 0 & 1 & 0 & 0 & 0 & 0 & 0 & 0 & 0 & 0 & 0 & 0 & … & 0 & 0 \\ 0 & 0 & 0 & … & 0 & 0 & 0 & 0 & 1 & … & 0 & 0 & 0 & 0 & 0 & 0 & 0 & 0 & 0 & 0 & 0 & 0 & … & 0 & 0 \\ 0 & 0 & 0 & … & 0 & 1 & 0 & 0 & 0 & … & 0 & 0 & 0 & 0 & 0 & 0 & 0 & 0 & 0 & 0 & 0 & 0 & … & 0 & 0 \\ 0 & 0 & 0 & … & 0 & 0 & 0 & 0 & 0 & … & 0 & 0 & 0 & 0 & 0 & 0 & 0 & 0 & 0 & 0 & 0 & 1 & … & 0 & 0 \\ 0 & 0 & 0 & … & 0 & 0 & 0 & 0 & 0 & … & 0 & 0 & 0 & 0 & 0 & 0 & 0 & 0 & 1 & 0 & 0 & 0 & … & 0 & 0 \\ 0 & 0 & 0 & … & 0 & 0 & 0 & 0 & 0 & … & 0 & 0 & 0 & 0 & 0 & 1 & 0 & 0 & 0 & 0 & 0 & 0 & … & 0 & 0\end{matrix} ]}^{T}$$

To eliminate the impact of the contaminant intrusion time on different complaint matrices, the position of non-zero elements is normalized according to the following formula, where Tᵢ₁ represents the original time subscript value of a non-zero element in the matrix, T(−)T(−)T(−)T̅ represents the original average value of time subscripts of non-zero elements in the matrix, and T_{i1,change} represents the changed time subscript value of a non-zero element. After the normalization, an average value of time subscripts of all non-zero elements is 24.

T_{i1,changed}=Tᵢ₁−T̅+24 (i1=1,2, . . . ,n)

The consumer complaints used in practically locating a contamination source are specifically pre-processed as follows:

For repeated complaints about a same node, the earliest complaint making time is used as the complaint making time for the node; and the complaint making time for all nodes is classified by a time step size of 30 min. For example, if a consumer makes a complaint at 9:20, it is approximately considered that the complaint is made at 9:30 since 20 min is more than 15 min; and if a consumer makes a complaint at 9:10, it is approximately considered that the complaint is made at 9:00 since 10 min is less than 15 min.

As the pattern recognition method, a convolutional neural network is used in the step 4), which has hyper-parameters as shown in Table 1. The program is run in a deep learning framework. The hyper-parameters for the training process are set as follows: the initial learning rate is 0.1; the attenuation coefficient is 0.99; L2 regularization is used in two fully connected layers at a regularization coefficient of 0.0001; and the number of training iterations is 15000.

In this embodiment, by taking the exemplary water distribution system Net3 as an example, the implementation process of the method for tracking and locating a contamination source in a water distribution system based on a convolutional neural network and consumer complaints will be further explained.

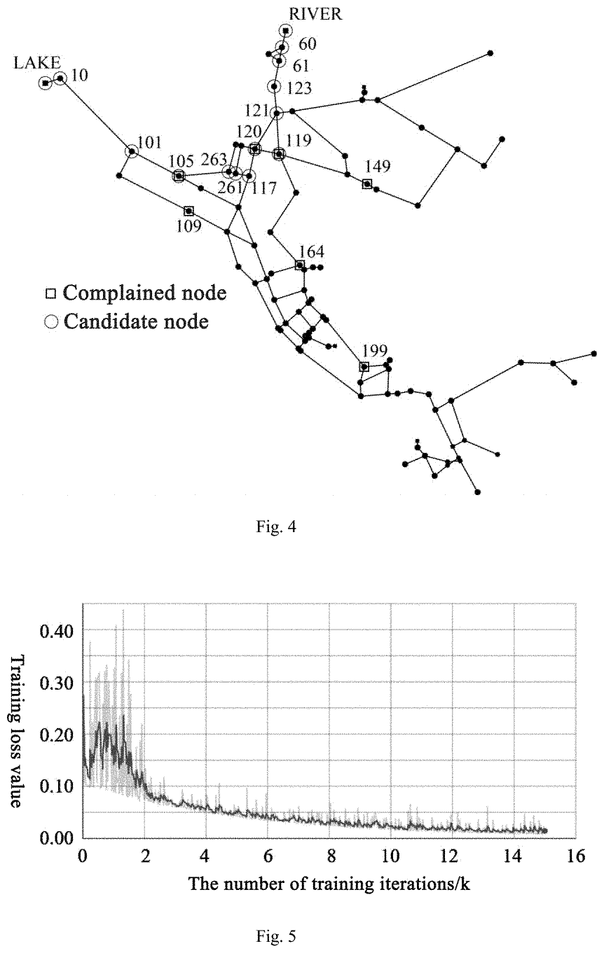

The model of the water distribution system contains 97 nodes (among which, there are two water source nodes and three water tower nodes), and 119 pipe segments. The topology structure of the water distribution system is shown in FIG. 4. In the present invention, it is assumed that the contaminant is conservative substance. That is, it diffuses with water, but it will not react with water. The mass concentration of the injected contaminants is ρ₀=25 mg/L, and the contaminants are continuously injected for 24 hours in a same mass concentration. The reporting limit RL of the human body to the contaminants is ρ_{L}=1.0 mg/L. During the simulation, the water power step size is set as 30 min and the water quality step size is set as 5 min.

1) A contamination matrix is generated by location information complained by consumers, and a set of candidate contamination source nodes is obtained.

It is assumed that, in a contamination event, consumers make complaints at nodes 105, 109, 120, 119, 149, 164 and 199, then the complaint making times are 6:40, 7:00, 8:20, 9:00, 11:40, 18:00, 12:20, respectively. The real contamination source is node 10. By improving the contamination matrix, the obtained set of candidate nodes is {10,60,61,101,105,117,119,121,123,261,263,Lake,River}.

2) By calculating the Chebyshev distance, similarity between contaminant diffusion patterns for the candidate nodes is determined and the candidate nodes are classified. The result of classification of candidate contamination source nodes is shown in the following table.

TABLE 1
Result of classification of candidate nodes

| Representative node | Node No. |
|---|---|
| Lake | 10, 101, Lake |
| 105 | 105, 120, 261, 263 |
| 117 | 117 |
| 121 | 119, 121, 123 |
| River | 60, 61, River |

3) A random complaint hysteresis time is added on the basis of the contaminant diffusion patterns and a consumer complaint sample is constructed.

During the water power and water quality simulations, it is assumed that the contamination occurs at 0:00, noisy samples are constructed and normalized. In this embodiment, the mean value μ used for constructing training samples is set as 3, with a standard deviation σ of 2.0. For each candidate node, 2800 samples are generated, including 2000 training samples and 800 validation samples. There are total 10000 training sets and 4000 validation sets. In the testing sets, it is assumed that Δt follows the normal distribution of different standard derivations. The standard derivations are uniformly distributed in an interval [1,4]. For each candidate contamination source type, 800 testing samples are generated.

4) The convolutional neural network is trained, validated and tested by the samples that are classified into a training set, a validation set and a testing set, to assess the validity of the model, and the convolutional neural network is used in tracking and locating a contamination source in a real contamination event.

At the beginning of training, the initial loss value is 0.4206. The model is well trained within a short period of time. After iterations, the loss value is decreased to 0.01457. The loss reduction curve during the training is shown in FIG. 5. As the number of iterations increases, the loss value overall shows a downward trend, gradually to 0. That is, the model exhibits strong convergence to the training samples. The trained model is called to perform cross validation. The obtained accuracy is 93.1%. it is indicated that the model has high prediction accuracy for samples in which the hysteresis time Δt follows the normal distribution. The test accuracy of the CNN model is shown in the third column of Table 2. It is shown, by the test results, that the established CNN model exhibits high recognition capability for complaint hysteresis times of different discrete degrees.

TABLE 2
Prediction accuracy for different normal distribution noises

| | | Prediction accuracy of the |
|---|---|---|
| | Normal distribution | convolutional neural network |
| Cross-validation | Δ~N(μ, 2.0²) | 0.931 |
| Test | Δ~N(μ, 1.0²) | 0.981 |
| | Δ~N(μ, 1.5²) | 0.959 |
| | Δ~N(μ, 2.5²) | 0.899 |
| | Δ~N(μ, 3.0² | 0.866 |
| | Δ~N(μ, 3.5²) | 0.826 |
| | Δ~N(μ, 4.0²) | 0.786 |

The real consumer complaint matrix is input to the trained CNN model to locate the contamination source. The probability of the candidate contamination sources being the real contamination source is 0.999, 7.563×10⁻⁵, 4.165×10⁻⁴, 1.815×10⁻⁵, 8.015×10⁻⁹, respectively. The result of prediction shows that the contamination source is in a set of nodes {10,101,Lake}. This coincides to the previously assumed contamination source location.

The above descriptions are merely specific implementations of the present invention. However, the protection scope of the present invention is not limited thereto. Various equivalent modifications and replacements may be conceived by a person of ordinary skill in the art within the technical scope disclosed in the present invention. Those modifications and replacements shall be included within the protection scope of the present invention. Therefore, the protection scope of the present invention shall be subject to the protection scope of the claims.

What is claimed is:

1. A method for tracking and locating a contamination source in a water distribution system based on consumer complaints, comprising following steps:
S1: generating a contamination matrix by location information complained by consumers;
S2: determining similarity between candidate nodes and classifying the candidate nodes;
S3: adding a random complaint hysteresis time and constructing a consumer complaint sample; and
S4: training, validating and testing a convolutional neural network by the consumer complaint sample, and using the convolutional neural network in practically tracking and locating a contamination source.

2. The method for tracking and locating a contamination source in a water distribution system based on consumer complaints according to claim 1, wherein the contamination matrix in the step S1 is expressed by the following formula:
$$C = [ \begin{matrix}{k}_{1 , 1} & … & {k}_{1 , n - 1} & {k}_{1 , n} \\ … & … & … & … \\ {k}_{m - 1 , 1} & … & {k}_{m - 1 , n - 1} & {k}_{m - 1 , n} \\ {k}_{m , 1} & … & {k}_{m , n - 1} & {k}_{m , n}\end{matrix} ]$$
where, C is the contamination matrix, m is the number of nodes in a water distribution system, n is the number of points complained by consumers, and k_{i,j}=0 or 1, wherein k_{i,j}=0 when contaminants are injected to the i^{th} node but the complained j^{th} node is not perceived as being contaminated, and k_{i,j}=1 when contaminants are injected to the i^{th} node and the complained j^{th} node is perceived as being contaminated, 1≤i≤m, 1≤j≤n.

3. The method for tracking and locating a contamination source in a water distribution system based on consumer complaints according to claim 1, wherein the determination and classification in the step S2 are done by the Chebyshev distance, expressed by the following formula:
D_{Chebyshev}(t′,T′)≤1
where, t′ and T′ each represent a relative time vector for contaminants added in two candidate contamination source nodes to reach complained nodes.

4. The method for tracking and locating a contamination source in a water distribution system based on consumer complaints according to claim 1, wherein the consumer complaint sample in the step S3 is a 48×n matrix containing elements 0 and 1.

5. The method for tracking and locating a contamination source in a water distribution system based on consumer complaints according to claim 4, wherein normalization of the 48×n matrix containing elements 0 and 1 is to normalize the position of non-zero elements so that an average value of time subscripts of all non-zero elements is 24, expressed by the following formula:
T_{i1,changed}=Tᵢ₁−T̅+24; (i1=1,2, . . . n)
where, Tᵢ₁ represents the original time subscript value of a non-zero element in the matrix, T_{i1,changed} represents the changed time subscript value of a non-zero element, and T̅ represents an average value of time subscripts of all non-zero elements.

6. The method for tracking and locating a contamination source in a water distribution system based on consumer complaints according to claim 1, wherein the convolutional neural network in the step S4 has hyper-parameters set as follows:

| Name | Structural parameter | Activation function |
|---|---|---|
| Input layer | 48 × n matrix | |
| Convolutional layer 1 | 3 × 3 × 8 S = 1 | ReLU |
| Convolutional layer 2 | 3 × 3 × 8 S = 1 | ReLU |
| Pooling layer | 2 × 2 S = 2 (Max pooling) | |
| Fully connected layer | 32 (neuron) | ReLU |
| Output layer | a | Softmax |

where, a represents the number of types of candidate nodes, and S represents the movement step size.

7. The method for tracking and locating a contamination source in a water distribution system based on consumer complaints according to claim 1, wherein the convolutional neural network in the step S4 has an initial learning rate of 0.1 and an attenuation coefficient of 0.99, uses L2 regularization in two fully connected layers at a regularization coefficient of 0.0001, and has a number of training iterations of 15000.
